(19) United States
(12) Patent Application Publication
(10) Pub. No.: US 2002/0016505 A1
(43) Pub. Date: Feb. 7, 2002

(54) Storage media for latent heat storage systems

(75) Inventors: Joachim Gally, Muhltal (DE); Ralf Glausch, Darmstadt (DE); Udo Heider, Riedstadt (DE); Natascha Lotz, Erzhausen (DE); Mark Neuschutz, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, Darmstadt (DE)

(21) Appl. No.: 09/835,816

(22) Filed: Apr. 17, 2001

(30) Foreign Application Priority Data
Apr. 17, 2000 (DE) 100 189 38.5

(51) Int. Cl. C07C 335/00

(52) U.S. Cl. 564/28; 534/570

(57) ABSTRACT

The present invention generally relates to compositions for storing heat energy in the form of heat of phase transition, and to their use. The compositions of the invention for storing heat comprise at least one heat storage material and at least one auxiliary and are characterized in that the composition comprises at least one heat storage material which has at least one solid/solid phase transition and is solid throughout the application range.

The present invention relates to compositions for storing thermal energy in the form of heat of phase transformation, and to their use.

In industrial processes it is a frequent necessity to avoid thermal peaks or deficits, i.e. thermostating is necessary. For this purpose it is common to use heat exchangers. These contain heat transfer media which transport the heat from one location or medium to another. In order to dissipate thermal peaks, for example, the emission of the heat via a heat exchanger to the air is utilized. This heat, however, is then no longer available to compensate thermal deficits. This problem is solved by the use of heat storage systems.

Examples of known storage media include water or stones/concrete, in order to store perceptible (“sensible”) heat, or phase change materials (PCMs) such as salts, salt hydrates or mixtures thereof, in order to store heat in the form of heat of fusion (“latent” heat).

It is known that the melting of a substance, i.e. its transition from the solid to the liquid phase, involves consumption, i.e. absorption, of heat which, for as long as the liquid state persists, is stored in latent form, and that this latent heat is released again on solidification, i.e. on transition from the liquid to the solid phase.

A fundamental requirement for the charging of a heat storage system is a higher temperature than can be obtained in the course of discharge, since heat transport/flux necessitates a temperature difference. The quality of the heat is dependent on the temperature at which it is available: the higher the temperature, the more diverse the uses to which the heat may be put. For this reason, it is desirable for the temperature level in the course of storage to fall as little as possible.

In the case of sensible heat storage (e.g. by heating of water) the input of heat is associated with gradual heating of the storage material (and vice versa during discharge), whereas latent heat is stored and discharged at the melting temperature of the PCM. Latent heat storage therefore has the advantage over sensible heat storage that the temperature loss is limited to the loss during heat transport from and to the storage system.

To date, the storage media used in latent heat storage systems have usually been substances which have a solid/liquid phase transition within the temperature range critical to the application, i.e. substances which melt during the application.

Accordingly, the literature discloses the use of paraffins as storage media in latent heat storage systems. International Patent Application WO 93/15625 describes shoe soles containing PCM microcapsules. The PCMs proposed comprise either paraffins or crystalline 2,2-dimethyl-1,3-propanediol and/or 2-hydroxymethyl-2-methyl-1,3-propanediol. Application WO 93/24241 describes fabrics with a coating containing such microcapsules and binders. In this case, it is preferred to use paraffinic hydrocarbons having 13 to 28 carbon atoms. European Patent EP-B-306 202 describes fibers having heat storage properties, the storage medium being a paraffinic hydrocarbon or a crystalline plastic and the storage material being integrated in the form of microcapsules into the fiber base material.

U.S. Pat. No. 5,728,316 recommends salt mixtures based on magnesium nitrate and lithium nitrate for storing and utilizing thermal energy. Heat storage in that case takes place in the melt above the melting temperature of 75.6° C.

In the case of the abovementioned storage media in latent heat storage systems, there is a transition to the liquid state during the application. This is associated with problems with regard to the technical use of the storage media in latent heat storage systems, since in principle there must be a sealing or encapsulation which prevents an emergence of liquid leading to loss of substance and/or contamination of the environment. Especially in the case of use in or on flexible structures, such as fibers, fabrics or foams, for example, this generally necessitates a microencapsulation of the heat storage materials: this, however, is often incomplete and/or technically very demanding, and hence expensive. For example, as described in Patent EP-B-306 202, it is preferred if these microcapsules have double walls.

Furthermore, there is a sharp rise in the vapor pressure of many potentially suitable compounds on melting, so that the volatility of the melts often opposes long-term use of the storage materials. The technical deployment of melting PCMs is frequently accompanied by problems owing to severe changes in volume during the melting of many substances.

There is therefore a need for storage media for latent heat storage systems whose use does not entail the abovementioned problems.

It has now surprisingly been found that certain substances which have a solid/solid transition in the application range are also suitable as heat storage materials. Since these substances remain solid throughout the application, there is no need for encapsulation. Accordingly, loss of the storage medium or contamination of the environment by the melt of the storage medium in latent heat storage systems can be ruled out.

### SUMMARY OF THE INVENTION

The present invention first provides, accordingly, a composition for storing heat, comprising at least one heat storage material and at least one auxiliary, characterized in that the composition comprises at least one heat storage material which has at least one solid/solid phase transition and is solid throughout the application range.

The invention secondly provides for the use of compounds which have at least one solid/solid phase transition as storage media in latent heat storage systems.

Advantages of these heat storage materials are primarily:

the solid state of the storage medium, with its greater ease of handling in comparison to liquids;

the small change in volume accompanying the phase transition, which permits insertion into complex structural components;

and the low vapor pressure of the heat-storing high-temperature phase.

The heat storage material preferably comprises a compound conforming to the empirical formula

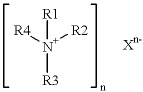

in which R1, R2, R3 and R4 each independently of one another are selected from the group consisting of the radicals H, C₁-C₃₀ alkyl and C₁-C₃₀ hydroxyalkyl and Xⁿ⁻ is selected from the group of the monoatomic and complex inorganic anions or from the group of the organic anions, with n resulting from the ionic charge of the anion. Preferred monoatomic inorganic anions used are anions from the group consisting of fluoride, chloride, bromide and iodide. Complex inorganic anions in the sense of the present invention are all anions which are composed of at least 2 different elements, preferably anions having a central atom and ligands; in particular, nitrate, chlorate, perchlorate, (hydrogen) sulfate, ((di-)hydrogen) phosphate, tetrachlorochromate, tetrachloromanganate, tetrachlorocadmate, tetrachloropalladate and tetrachloroferrate should be mentioned here. The organic anions used in particular are anions of the organic acids, such as formate, acetate, propionate, butyrate, caprate, stearate, palmitate, acrylate, oleate, oxalate, malonate, succinate, glutarate, benzoate, 2-nitrobenzoate, salicylate and phenylacetate.

Because of their favorable transition temperatures and high transition enthalpies, fields of use of these compounds are located within the area of thermostating, so that the present invention additionally provides for the use of the abovementioned compounds for thermostating. Thermostating in the sense of the present invention means both the thermal insulation and thus constant holding of a temperature and the buffering of short-term temperature fluctuations or temperature peaks. Applications may consist both in heat storage and controlled release and in uptake of heat and, in connection therewith, cooling.

Preferred heat storage materials in this context are those comprising a compound which in its low-temperature form crystallizes in a sheetlike perovskite type. Among these compounds, preference is given in turn, in accordance with the invention, to the monoalkylammonium tetrachlorochromates, mono-alkylammonium tetrachloromanganates, monoalkylammoniumtetrachlorocadmates, monoalkylammoniumtetrachloropalladates and monoalkylammonium tetrachloroferrates with alkyl chain lengths from the range C₁-C₃₀. Particular preference is given to the abovementioned monoalkylammonium tetrachlorometallates having C₁, C₂, C₄, C₆, C₈, C₁₀, C₁₂, C₁₄, C₁₆ or C₁₈ alkyl chains. Physical properties of these compounds are described, for example, in the publications G. F. Needham, R. D. Willett, H. F. Franzen, J. Phys.-Chem. 88 (1984) 674 and W. Depmeier, Ferroelectrics 24 (1981) 81.

Another class of heat storage materials particularly preferred in accordance with the invention comprises dialkylammonium salts. It is preferred to use those dialkylammonium salts whose radicals R1 and R2 have equal carbon chain lengths and in which the radicals R3 and R4 are hydrogen. These dialkylammonium salts may be used in pure, crystalline form. However, in particular in order to set transition temperatures in a targeted manner, it may also be desirable to use mixed crystals of different dialkylammonium salts.

The heat storage materials particularly preferred in accordance with the invention include the symmetric dialkylammonium salts, e.g.: of the following group: diethylammonium chloride, dipropylammonium chloride, dibutylammonium chloride, dipentylammonium chloride, dihexylammonium chloride, dioctylammonium chloride, didecylammonium chloride, didodecylammonium chloride, dioctadecylammonium chloride, diethylammonium bromide, dipropylammonium bromide, dibutylammonium bromide, dipentylammonium bromide, dihexylammonium bromide, dioctylammonium bromide, didecylammonium bromide, didodecylammonium bromide, dioctadecylammonium bromide, diethylammonium nitrate, dipropylammonium nitrate, dibutylammonium nitrate, dipentylammonium nitrate, dihexylammonium nitrate, dioctylammonium nitrate, didecylammonium nitrate, dioctylammonium chlorate, dioctylammonium acetate, dioctylammonium formate, didecylammonium chlorate, didecylammonium acetate, didecylammonium formate, didodecylammonium chlorate, didodecylammonium formate, didodecylammonium hydrogensulfate, didodecylammonium propionate, dibutylammonium-2-nitrobenzoate, diundecylammonium nitrate and didodecylammonium nitrate. The physicothermal characterization of the dialkylammonium chlorides can be found in the publication M. J. M. van Oort, M. A. White, Ber. Bunsenges. Phys. Chem. 92 (1988)168. Which compound is best suited to a specific case depends primarily on the field of use of the latent heat storage systems. In general, however, the dialkylammonium salts with high transition enthalpies are particularly preferred. Particular mention may be made here of dioctylammonium chloride, didecylammonium chloride, didodecylammonium chloride, dioctadecylammonium chloride, dihexylammonium bromide, didecylammonium bromide, didodecylammonium bromide, dioctadecylammonium bromide, dihexylammonium nitrate, dioctylammonium nitrate, didecylammonium nitrate, dioctylammonium chlorate, dioctylammonium acetate, dioctylammonium formate, didecylammonium chlorate, didecylammonium acetate, didecylammonium formate, didodecylammonium chlorate, didodecylammonium formate, didodecylammonium hydrogensulfate, didodecylammonium propionate, dibutylammonium-2-nitrobenzoate and didodecylammonium nitrate.

For applications in the field of thermostatic clothing, such as winter coats or ski jackets or shoes, for example, it is advantageous, for example, that the transition temperatures lie below the body temperature and well above the frost limit. The same requirements must be met by compounds suitable for the thermal conditioning of buildings. For applications of this kind, particularly preferred dialkylammonium salts are dioctylammonium chloride, dihexylammonium bromide, dioctylammonium bromide and dihexylammonium nitrate.

Furthermore, on the basis of its transition temperature of 11° C., dihexylammonium nitrate is outstandingly suitable for applications where slight cooling is necessary, while the compounds with transition temperatures below 0° C. are suitable for cooling media which are intended to maintain temperatures below the freezing point of water. For industrial heat storage, or for keeping meals warm, suitable compounds are those, in turn, which have a transition temperature in the range from 50° C. to below 100° C. Of particular advantage in this context are the dialkylammonium chlorides, bromides and nitrates having alkyl chains of at least 10 carbon atoms in length.

A further important factor for the application of the storage media in latent heat storage systems is that the transition enthalpy does not fall below a certain energy minimum, since otherwise the amounts of substance needed to store the energy become too great. In accordance with the invention it is preferred, therefore, if the heat storage material has a solid/solid phase transition in the application range that has an enthalpy of at least 50 J/g, preferably of at least 80 J/g, and with particular preference of at least 150 J/g. In this context, the enthalpies of the solid/solid phase transitions, which are often lower than customary heats of fusion, appear at first glance to be a disadvantage of these substances in comparison to the melting PCMs. Since, however, such melting PCMs are used in encapsulated form, especially in microencapsulated form, it is necessary for the enthalpy per gram of substance used to take account of the encapsulation material as well.

Since it is important for the energy yield and for the rapid uptake and release of energy that the heat storage material has a large surface area and/or is finely distributed in a medium/auxiliary, it is of advantage in accordance with the invention if the heat storage material has an average crystallite size in the range from 0.1 to 1000 μm, preferably in the range from 1 to 100 μm.

For the majority of end uses of latent heat storage systems, it is further of advantage if the storage material is insoluble in water, since in that case moisture exposure, during washing or as a result of rain, for example, does not lead to losses of substance.

As already mentioned earlier on above, it is preferable depending on end-use application for the composition for storing heat to exhibit certain transition temperatures. Normally, the application range of the storage media of the invention in latent heat storage systems is situated within the temperature range between −100° C. and 150° C., generally in the temperature range from −50° C. to 100° C., and usually in fact in the temperature range from 0° C. to 90° C. Accordingly, it is preferable for the compositions of the invention to comprise heat storage materials which have a solid/solid phase transition within these temperature ranges.

Besides the heat storage material itself, the compositions of the invention for storing heat comprise at least one auxiliary, preferably inert. In one preferred embodiment of the invention, the said at least one auxiliary comprises a substance or preparation having good thermal conductivity, in particular a metal powder, metal granules or graphite. The heat storage material is preferably in a state of intimate mixture with the auxiliary, the overall composition preferably being in the form either of a loose bed or of shaped bodies. By shaped bodies are meant, in particular, all structures which can be produced by compacting methods, such as pelletizing, tableting, roll compacting or extrusion. The shaped bodies may adopt any of a very wide variety of three-dimensional forms, such as spherical form, cube form or rectangular block form. In a further particularly preferred embodiment, the mixtures or shaped bodies described herein comprise paraffin as an additional auxiliary. Paraffin is used in particular when for the application the intention is to produce intimate contact between the heat storage composition and a structural component because, generally, as the paraffin melts, air displaces at the contact faces ensuring close contact between the heat storage material and the structural component. For example, it is possible in this way to incorporate latent heat storage systems with a precision fit for the cooling of electronic components. In connection with the assembly of the heat storage systems, the handling in particular of a shaped body described above is simple: during the application, the paraffin melts, displaces air at the contact faces, and so ensures close contact between heat storage material and component. Preferably, therefore, compositions of this kind are used in devices for cooling electronic components.

In a likewise preferred embodiment of the invention the at least one auxiliary comprises a binder, preferably a polymeric binder. In this case the crystallites of the heat storage material are preferably in a state of fine distribution in the binder. The heat storage compositions may then be in the form of fibers, in which case the binder acts simultaneously as fiber base material and is preferably a synthetic polymer. In accordance with the invention, fibers which comprise the heat storage material may also be of such construction that a natural or synthetic fiber forms the basic structure of the fiber and the binder or binders together with the heat storing material form a coating around this fiber. These fibers may then be used to obtain fabrics having thermostatic properties. Another way of obtaining heat storing fabrics of this kind is by coating a ready-made fabric with the composition comprising heat storage medium and binder. In accordance with the invention, a coating of this kind may also be present on another surface.

The preferably polymeric binders which may be present may comprise any polymers which are suitable as binders according to the end-use application. The polymeric binder is preferably selected from curable polymers or polymer precursors which in turn are preferably selected from the group consisting of polyurethanes, nitrile rubber, chloroprene, polyvinyl chloride, silicones, ethylene-vinyl acetate copolymers and polyacrylates. The person skilled in this art is well aware of how the heat storage materials are appropriately incorporated into these polymeric binders. It causes him or her no difficulty to find, if necessary, the requisite additives, such as emulsifiers, for example, which stabilize such a mixture.

In a further variant of the invention, the compositions for storing heat are in the form of an open-celled or closed-celled foam, the auxiliary, which is preferably a polymer, forming the matrix of the foam in which the crystallites of the heat storage material are present in a state of fine distribution. Foams of this kind may be used for thermal insulation and, preferably, for imparting thermostatic properties to clothing. The foams may either be applied on fabric layers or incorporated between fabric layers. Also conceivable is the direct use of the foams, for example as shoe soles. Such thermostatic clothing may then be used for a very wide variety of purposes. Improved heat regulation in comparison to conventional winter clothing is only one advantageous field of application. Another promising application is that of protective clothing for fire fighters, for example, which absorbs heat peaks and so protects against burns.

In a likewise preferred variant of the invention, the binder comprises an inorganic binder based on water-insoluble silicates, phosphates, sulfates or metal oxides, preferably cement or plaster. One use of such compositions, preferred in accordance with the invention, is in the thermostating of buildings. In this case, either the building material may be formed directly of the composition of the invention, for heat storage, or the heat storage composition may be incorporated into the building material or coatings of the building material.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosure of applications, patents and publications, including DE 100 18 938.5 filed Apr. 17, 2000, cited above or below, is hereby incorporated by reference.

### EXAMPLES

### Example 1

Solid/solid phase transition measurements were conducted for a variety of solid/solid phase change materials. The solid/liquid phase transitions (melting point) were also measured. The results are compiled in the table below.

TABLE 2
Examples of solid/solid and solid/liquid phase transitions

| | | Heating | Heating | Cooling | Cooling | Sub- | Melting |
|---|---|---|---|---|---|---|---|
| Amine | Acid | onset | enthalpy | onset | enthalpy | cooling | point |
| Dihexylamine | Hydrogen chloride | 6° C. | 51 J/g | 3° C. | 51 J/g | 3° C. | >100° C. |
| Dihexylamine | Nitric acid | 10° C. | 110 J/g | −8° C. | 99 J/g | 18° C. | >100° C. |
| Dioctylamine | Chioric acid | 14° C. | 112 J/g | 14° C. | 122 J/g | 0° C. | 37° C. |
| Dihexylamine | Hydrogen bromide | 19° C. | 72 J/g | 14° C. | 71 J/g | 5° C. | >100° C. |
| Dioctylamine | Hydrogen chloride | 21° C. | 87 J/g | 19° C. | 74 J/g | 2° C. | >100° C. |
| Dioctylamine | Hydrogen bromide | 29° C. | 79 J/g | 27° C. | 79 J/g | 2° C. | >100° C. |
| Dioctylamine | Acetic acid | 36° C. | 177 J/g | 20° C. | 163 J/g | 16° C. | 40° C. |
| Dioctylamine | Nitric acid | 44° C. | 154 J/g | 26° C. | 144 J/g | 18° C. | >100° C. |
| Dioctylamine | Formic acid | 45° C. | 145 J/g | 17° C. | 127 J/g | 28° C. | >100° C. |
| Didecylamine | Hydrogen chloride | 49° C. | 117 J/g | 43° C. | 113 J/g | 5° C. | >100° C. |
| Didecylamine | Chloric acid | 54° C. | 140 J/g | 41° C. | 131 J/g | 13° C. | >100° C. |
| Didodecylamine | Chloric acid | 54° C. | 168 J/g | 47° C. | 155 J/g | 6° C. | >100° C. |
| Didodecylamine | Formic acid | 56° C. | 156 J/g | 45° C. | 145 J/g | 11° C. | 87° C. |
| Didecylamine | Hydrogen bromide | 56° C. | 102 J/g | 50° C. | 100 J/g | 6° C. | >100° C. |
| Didecylamine | Nitric acid | 57° C. | 153 J/g | 44° C. | 149 J/g | 13° C. | >100° C. |
| Didecylamine | Acetic acid | 58° C. | 151 J/g | 53° C. | 140 J/g | 5° C. | 68° C. |
| Didodecylamine | Acetic acid | 64° C. | 178 J/g | 63° C. | 163 J/g | 1° C. | 76° C. |
| Didodecylamine | Sulfuric acid | 64° C. | 50 J/g | 61° C. | 49 J/g | 3° C. | 97° C. |
| Didodecylamine | Hydrogen chloride | 65° C. | 132 J/g | 60° C. | 127 J/g | 5° C. | >100° C. |
| Dibutylamine | 2-Nitrobenzoic acid | 66° C. | 45 J/g | 41° C. | 40 J/g | 25° C. | 118° C. |
| Didodecylamine | Propionic acid | 66° C. | 169 J/g | 66° C. | 164 J/g | 1° C. | 73° C. |
| Didecylamine | Formic acid | 67° C. | 161 J/g | 46° C. | 148 J/g | 21° C. | 79° C. |
| Didodecylamine | Nitric acid | 69° C. | 160 J/g | 62° C. | 161 J/g | 7° C. | >100° C. |
| Didodecylamine | Hydrogen bromide | 78° C. | 124 J/g | 65° C. | 119 J/g | 6° C. | >100° C. |

### Measurement Conditions

a) Differential Scanning Calorimetry (DSC): Mettler Toledo, 2-10 mg of sample in a hermetically sealed aluminium crucible, measurement cycle: room temperature (RT) to 120° C. to −50° C. to RT for 5 cycles (4th and 5th cycle evaluated), heating and cooling rate 5 K/min

b) Melting point: Büchi melting point apparatus, temperature range 30 to 100° C., heating rate 10 K/min

### Example 2

### Production of Pressings

The active material didodecylammonium chloride (01/EX16), on its own or together with the corresponding graphite component KS6, was ground on a laboratory mill from 1 ka. The grinding duration was 2×30 seconds.

TABLE 3
Sample preparation and designation

| Starting substance | Amount | Remarks | New designation |
|---|---|---|---|
| 01/EX16 alone | 1 g | no grinding | 01/EX/16 |
| 01/EX/16 + | 4 g | 2 g each were ground | 01/NP/2.1 |
| 10% graphite | | simultaneously | |
| 01/EX/16 alone | 2 g | ground | 01/NP/2.2 |

0.5 g was weighed out in each case and introduced into the entry aperture of the pressing mould. Using the manual lever, a pressure of 5 t was applied. This pressure was maintained for 1 min, with adjustment if necessary.

Experiments with a higher pressure were also conducted. 2.5 g of material (01/NP/2.1) were pressed at a pressure of 20 t for 1 min.

TABLE 4
Pressings

| | Initial | | | |
|---|---|---|---|---|
| Sample No. | weight | Height | Diameter | Remarks |
| 01/EX/16 | 1.005 g | 0.5 cm | 1.6 cm | Particles are visible, no |
| | | | | uniform pressing, stable |
| 01/NP/2.1 | 0.5036 g | 0.2 cm | 1.6 cm | very stable pressing, |
| | | | | smooth surface |
| 01/NP/2.1 | 0.4993 g | 0.2 cm | 1.6 cm | very stable pressing, |
| | | | | smooth surface |
| 01/NP/2.2 | 0.5002 g | 0.2 cm | 1.6 cm | very stable pressing, |
| | | | | smooth surface |
| 01/NP/2.1 | 2.4200 g | 0.2 cm | 4.0 cm | very stable pressing, |
| | | | | smooth surface |

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A composition for storing heat, comprising at least one heat storage material and at least one auxiliary for aiding heat transmission, wherein at least one of the at least one heat storage material has at least one solid/solid phase transition and is solid throughout the application range.

2. A composition for storing heat according to claim 1, wherein one heat storage material comprises a compound conforming to the empirical formula 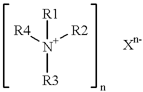
wherein R1, R2, R3 and R4 are each, independently, a radical H, C₁-C₃₀ alkyl or C₁-C₃₀ hydroxyalkyl, and Xⁿ⁻ is a monoatomic or complex inorganic anion, where n results from the ionic charge of the anion:

3. A composition for storing heat according to claim 1, wherein one heat storage material comprises a compound wherein its low-temperature form crystallizes in a sheetlike perovskite type.

4. A composition for storing heat according to claim 2, wherein one heat storage material comprises a dialkylammonium salt.

5. A composition for storing heat according to claim 2, wherein one heat storage material comprises mixed crystals of different dialkylammonium salts.

6. A composition for storing heat according to claim 2, wherein one heat storage material comprises diethylammonium chloride, dipropylammonium chloride, dibutylammonium chloride, dipentylammonium chloride, dihexylammonium chloride, dioctylammonium chloride, didecylammonium chloride, didodecylammonium chloride, dioctadecylammonium chloride, diethylammonium bromide, dipropylammonium bromide, dibutylammonium bromide, dipentylammonium bromide, dihexylammonium bromide, dioctylammonium bromide, didecylammonium bromide, didodecylammonium bromide, dioctadecylammonium bromide, diethylammonium nitrate, dipropylammonium nitrate, dibutylammonium nitrate, dipentylammonium nitrate, dihexylammonium nitrate dioctylammonium nitrate, didecylammonium nitrate, diundecylammonium nitrate, didodecylammonium nitrate, dioctylammonium chlorate, dioctylammonium acetate, dioctylammonium formate, didecylammonium chlorate, didecylammonium acetate, didecylammonium formate, didodecylammonium chlorate, didodecylammonium formate, didodecylammonium hydrogensulfate, didodecylammonium propionate, or dibutylammonium-2-nitrobenzoate.

7. A composition for storing heat according to claim 1, wherein the heat storage material has an average crystallite size of about 0.1 to about 1000 μm, and the material is insoluble in water.

8. A composition for storing heat according to claim 1, wherein the application range of the heat storage material has a solid/solid phase transition which has an enthalpy of at least about 50 J/g.

9. A composition for storing heat according to claim 1, wherein the application range of the heat storage material has a solid/solid phase transition which lies within the temperature range of about −100° C.—about 150° C.

10. A composition for storing heat according to claim 1, wherein the at least one auxiliary comprises a substance or preparation having good thermal conductivity in the form of a loose bed or in the form of shaped bodies.

11. A composition for storing heat according to claim 10, wherein the auxiliary comprises paraffin.

12. A composition for storing heat according to claim 1, wherein the at least one auxiliary comprises a binder finely distributed with the crystallites of the heat storage material.

13. A composition for storing heat according to claim 12, wherein the composition is in the form of fibers, with the binder serving simultaneously as fiber base material.

14. A composition for storing heat according to claim 12, wherein the composition is in the form of fibers, with a natural or synthetic fiber forming the basic structure of the fiber and the binder or binders together with the heat storage material forming a coating around this fiber.

15. A composition for storing heat according to claim 12, wherein the composition is in the form of a coating on a surface or around a textile fabric.

16. A composition for storing heat according to claims 12, wherein the polymeric binder is a curable polymer or polymer precursor polyurethane, a nitrile rubber, chloroprene, polyvinyl chloride, a silicone, an ethylene-vinyl acetate copolymer or a polyacrylate.

17. A composition for storing heat according to claim 1, wherein the composition is present in the form of an open-celled or closed-celled foam, with the auxiliary.

18. A composition for storing heat according to claim 12, wherein the binder comprises an inorganic binder comprising a water-insoluble silicate, a phosphate, a sulfate or a metal oxide.

19. A storage media for a latent heat storage system comprising a compound having at least one solid/solid phase transition.

20. A storage media according to claim 19 wherein the compound has the formula: 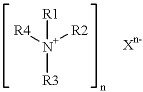
wherein R1, R2, R3 and R4 are each, independently of one another, a radical H, C₁-C₃₀ alkyl or C₁-C₃₀ hydroxyalkyl and Xⁿ⁻ is a monoatomic or a complex inorganic anion, wherein n results from the ionic charge of the anion.

21. A thermostating process comprising storing heat in a compound of the formula: 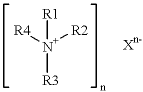
wherein R1, R2, R3 and R4 are each, independently of one another, a radical H, C₁-C₃₀ alkyl or C₁-C₃₀ hydroxyalkyl and Xⁿ⁻ is a monoatomic or a complex inorganic anion, wherein n results from the ionic charge of the anion.

22. A foam comprising a composition according to claim 17 for imparting thermostatic properties to clothing.

23. A device for cooling an electronic component comprising a composition according to claim 10.

24. A building comprising a composition according to claim 18 for the thermostating.

25. A composition according to claim 2 wherein Xⁿ⁻ is fluoride, chloride, bromide, iodide, nitrate, chlorate, perchlorate, sulfate, phosphate, tetrachlorochromate, tetrachloromanganate, tetrachlorocadmate, tetrachloropalladate, tetrachloroferrate, formate, acetate, propionate, butyrate, caprate, stearate, palmitate, acrylate, oleate, oxalate, malonate, succinate, glutarate, benzoate, 2-nitrobenzoate, salicylate or phenyl-acetate.

26. A composition according to claim 3 wherein the compound is a monoalkylammonium tetrachlorochromate, a monoalkylammonium tetrachloromanganate, a monoalkylammonium tetrachlorcadmate, a monoalkylammonium tetrachloropalladate, or a monoalkylammonium tetrachloroferrate having an alkyl chain length of C₁-C₃₀.

27. A composition according to claim 4 wherein R1 and R2 have identical carbon chain lengths and R3 and R4 are hydrogen.

28. A composition according to claim 6 wherein the one heat storage material comprises dioctylammonium chloride, didecylammonium chloride, didodecylammonium chloride, dioctadecylammonium chloride, dihexylammonium bromide, didecylammonium bromide, didodecylammonium bromide, dioctadecylammonium bromide, dihexylammonium nitrate, dioctylammonium nitrate, didecylammonium nitrate, dioctylammonium chlorate, dioctylammonium acetate, dioctylammonium formate, didecylammonium chlorate, didecylammonium acetate, didecylammonium formate, didodecylammonium chlorate, didodecylammonium formate, didodecylammonium hydrogensulfate, didodecylammonium propionate, dibutylammonium-2-nitrobenzoate, or didodecylammonium nitrate.

29. A composition according to claim 10 where the auxiliary comprises a metal powder or a metal granule or graphite, wherein the heat storage material is mixed with the auxiliary.

30. A composition according to claim 12 wherein the polymeric binder is a polyurethane, a nitrile rubber, chloroprene, polyvinyl chloride, a silicone, an ethylene-vinyl acetate copolymer or a polyacrylate.

31. A composition according to claim 2, wherein Xⁿ⁻ is an organic ion.
